(19) United States
(12) Patent Application Publication
(10) Pub. No.: US 2025/0313876 A1
(43) Pub. Date: Oct. 9, 2025

(54) ISOLATION OF DNA FRAGMENTS

(71) Applicant: Ribbon Bio GmbH, Vienna (AT)

(72) Inventors: Giulio Bernardinelli, Vienna (AT); Ishita Gupta, Vienna (AT); Gerald Wimsberger, Vienna (AT); Rodrigo Redondo, Vienna (AT)

(21) Appl. No.: 19/170,914

(22) Filed: Apr. 4, 2025

Related U.S. Application Data
(60) Provisional application No. 63/575,675, filed on Apr. 6, 2024.

(51) Int. Cl. C12P 19/34

(52) U.S. Cl. CPC C12P 19/34

(57) ABSTRACT

The present invention allows for the isolation of sequence correct DNA fragments from heterogeneous mixtures of fragments through the implementation of several steps for the purification of DNA molecules, the amplification of said DNA molecules, identification of sequence correct DNA fragments, and isolation of fragments as templates for PCR-based production of further downstream fragments.

### TECHNICAL FIELD

The present disclosure relates to methods of isolating sequence correct DNA fragments from heterogeneous mixtures of fragments.

### BACKGROUND

Artificial synthesis of polynucleotides can be achieved through “assembly methods”, which consist of biochemically joining oligonucleotides and polynucleotides of different sizes and of varying sequences in specific ways in order to obtain a larger molecule that has the desired target sequence. The common theme in building DNA molecules of thousands of base pairs is to chemically or enzymatically synthesize small fragments of up to few hundred nt or bp and then concatenate these together by cloning, ligation, polymerase cycling assembly (PCA), or Gibson assembly.

The “Gibson Assembly” is a method for linking several linear double stranded (ds) DNA fragments (size ranging from about 30 bp up to several Kbp). The method consists of joining many ds DNA fragments that have pairwise overlapping sequence homology. The overlapping homology region between fragments can range between about 15 to 80 bp. No overhangs (single stranded portions on a DNA duplex) are necessary, since the enzymatic machinery of the method produces said overhangs, fills in any remaining gaps and ligates the fragments. This enzymatic machinery makes use of three enzymes: T5 exonuclease, Phusion DNA polymerase and Taq DNA ligase, all in an isothermal reaction. The method is simple and versatile and can produce both linear and circular ds DNA products. This method, suffers, however from sequence constraints, for example, with homologous regions or repeats in the joined fragments.

In conventional approaches, amplification products are cloned into minimal DNA vectors and transformed into bacteria, to obtain clonal fragments for quality control (e.g., sequencing), as well as serving as templates for another round of PCR based amplification of sequence correct clones for downstream assembly purposes. While this procedure is relatively fast (e.g., overnight bacterial growth to obtain clonally derived colonies), this step cannot be further shortened, and bacteria provide several disadvantages for the selection of fragments. For example, to reduce the impact of potentially toxic gene fragments commercial strains of bacteria have to be used and potentially have to be grown at lower temperatures (e.g., increasing incubation/growth periods). In addition to these time constraints, there are other limitations of cloning-based approaches, for instance, bacteria may potentially recombine or alter cloned DNA fragments depending upon the sequence of inserts. Cloning, as well as handling of bacteria, is time, labour, and cost intensive and automation, whenever possible, requires the purchase of expensive specialized equipment. Cloning of potentially dangerous DNA fragments into bacteria is problematic from a regulatory perspective. DNA produced using bacteria is ill-suited for several downstream applications due to potential impurities derived from bacteria, which are either immunogenic or immunostimulatory, which is especially relevant in producing DNA or RNA based vaccines.

Conventional methods of assembling multi-kilobase polynucleotides composed of multiple kilobase fragments, requires two custom primers or pairs of primers (e.g., forward and reverse) for PCR amplification of each fragment. However, primer design, despite being a routine step in DNA amplification protocols, is a sub-optimal procedure because it is sequence-dependent and poses limitations for automating and scaling DNA assembly. Designing these primers requires meeting specific chemical-physical criteria such as length, melting temperature, and GC % tailored to the target sequence. Designing effective primers is non-trivial, and sub-optimal primers often lead to unexpected by-products dominating the amplification reaction, necessitating labor intensive purification, ill-suited for automation, thereby affecting scalability. Because of these limitations in primer design, achieving a standardized amplification protocol for all DNA fragments becomes arduous due to differing sequences and the risk of primer-induced mutations affecting sequence verified molecules' quality and delaying production. Additional problems are connected to the joining of single molecules. Intrinsic sequence features (e.g., repeats, secondary structures, GC-rich stretches) could hinder standard assembly procedures (e.g., “Gibson assembly”) and the sequence content cannot be joined using sticky ends if a restriction site used for generating cohesive ends is present within an “undomesticated” sequence (e.g., “Golden gate protocol”).

Accordingly, there is an unmet need for improved DNA assembly methods.

### SUMMARY

The present invention provides improved methods and tools for synthesizing polynucleotides.

The present invention allows for the isolation of sequence-correct DNA fragments from heterogeneous mixtures of fragments through the implementation of several steps for the purification of DNA molecules, the amplification of said DNA molecules, identification of sequence correct DNA fragments (e.g., by Next-generation sequencing (NGS)), and isolation of fragments (e.g., using magnetic beads) as template for PCR-based production of further downstream fragments.

DNA assembly refers to a molecular cloning method that physically links together multiple fragments of DNA, in an end-to-end fashion, to achieve a designed, longer DNA construct. This process allows the construction of novel biological systems and devices using defined components. These techniques are carried out in vitro and are typically enzymatically-driven with the final constructions being maintained in microbial host cells.

The present invention provides methods for cell-free assembly of nucleic acids. In a preferred embodiment, methods comprise assembling DNA fragments to provide a DNA assembly, attaching barcode caps, which may be linear caps that are end-protected by chemical means (e.g., phosphorothioate modification), amplifying the fragments to form an amplification product, and separating a target product from non-target materials. In general, fragment enrichment and/or purification according to the invention includes attaching linear caps to sequence assembly products at one or more point during the sequence assembly process. In one aspect, methods include either the addition of caps in tiered assemblies or the addition of linear caps during later stages of assembly via, for example, a digestion/ligation process as described below. According to methods of the invention, linear caps are designed to allow for the universal amplification of sequence assembly products, the selective assembly of subsets of sequences (e.g., via barcoding), and the amplification of sequence subsets with primers introducing selected features (e.g., methylation and other epigenetic features). In addition, linear caps are amenable to circularization or concatenation using DNA modifying enzymes (e.g., integrases, Teln, and others). In some embodiments, methods of the invention include clonal amplification, allowing for the identification of sequence correct molecules (clones) with adaptor elements that service as barcodes for the amplification process.

In some embodiments, hairpin oligos are used instead of linear caps to form a covalently closed DNA circle as the barcode cap. The present invention further provides methods for cell-free assembly of nucleic acids. Preferred methods comprise assembling DNA fragments to provide a plurality of longer DNA assemblies which can include mis-assembled DNA, attaching hairpin oligos to each of the plurality of DNA assemblies to form covalently closed DNA circles (including a covalently closed mis-assembled DNA circle), removing the mis-assembled DNA circle, amplifying the DNA circles to form amplification products, and separating target products from non-target materials. The hairpin oligos may include a barcode or may serve as a priming site for amplification (e.g., RCA). The mis-assembled DNA assembly may further comprise a DNA assembly comprising bulges due mis-paired bases or insertions/deletions. For example, misalignment may occur if the oligo is truncated (e.g., missing bases somewhere in the middle). Methods may further comprise nicking the bulges with an enzyme, wherein the enzyme is an endonuclease. The mis-assembled DNA may comprise incomplete DNA target sequences. Methods may further comprise a separating step, wherein the separating step comprises (i) capturing, using oligonucleotide probes, the target product onto magnetic beads, or (ii) capturing, using oligonucleotide probes, the target product onto a solid substrate and washing the solid substrate. The probes can contain any mechanism for binding to a solid support and may comprise click chemistries, biotin, and the magnetic beads may comprise streptavidin. The probes may comprise glycoproteins, and the magnetic beads may comprise lectin. The probes may comprise antigens, and the magnetic beads may comprise antibodies that bind the antigens. The amplifying step may comprise Rolling Circle Amplification (RCA). The method may further comprise sequencing the amplification products after the amplifying step. In one embodiment, methods of the invention comprise Amplification with a primer containing the forward strand (concatenates of the sequence), yielding only one long molecule of the target sequence followed by Synthesis of complementary molecules, each primed from the concatenate (and therefore containing the reverse sequence of the hybridization seq attached to the RCA primer). Each long forward concatenate would therefore allow the production of many reverse molecules that are used for sequencing purposes to select the right hybridization probe or combinations of hybridization probes.

In certain aspects, methods of the invention comprise including self-complementary DNA oligonucleotides into a fragment assembly to obtain DNA fragments that are capped by hairpin structures. DNA barcodes may further be incorporated into the hairpins to allow for the identification of fragments (e.g., unique molecular identifiers or UMI). In certain aspects, methods of the invention may include enzymatic introduction of nicks at positions where “bulges” in the DNA are present due to mis-ligation (using, for example, mismatch endonuclease I or Authenticase™). In addition, mismatch recognition domains may be used, to allow for labelling of mismatch containing fragments or their removal by using solid support coupled mismatch binding proteins or their mismatch recognizing domains. Methods may further comprise purifying DNA fragments e.g., with exonuclease digest to remove incomplete assembly products (e.g., assembly intermediates) as well as nicked molecules to increase the percentage of correctly assembled molecules.

Methods of the invention may further comprise sequencing of the amplification products to identify molecules that are sequence-correct. Methods may further comprise solid-phase coupling, as, for example, hybridizing the amplification products to a biotinylated DNA probe that can be captured with magnetic Streptavidin beads to remove all un-hybridized molecules with washing steps. The inventors also contemplate other methods of coupling to a solid support and biotin-streptavidin is presented as one of many examples apparent to the skilled artisan. The probe used for hybridization may consist of a biotinylated DNA oligo (optionally containing locked nucleic acids), annealed to an oligo that contains a constant region (complementary to the biotinylated molecule) and an overhang, which is sequence complementary to the DNA sequence attached to the primer for Rolling Circle Amplification.

As discussed above, the current invention also relates to the design of the terminal 5′ and 3′ hairpin oligonucleotides for attaching to the DNA assembly to form a covalently closed DNA circle. The terminal 5′ and a terminal 3′ oligonucleotides of a fragment are designed in such a way to have no free 5′ or 3′ ends. This could be obtained by a self-annealing oligonucleotide.

These hairpin oligonucleotides may further comprise accessory sequences such as a target site for universal PCR primers, attachment moieties such as biotin, or barcoding sequences (e.g., UMI).

The invention provides effective methods for enzymatic clean-up of the assembly products that confer exonuclease resistance to only covalently closed (e.g., fully assembled) assembly products. The invention further provides a method that prevents the involvement of the molecules' ends in undesired ligations and allows for various attachment chemistries and universal sequences to be embedded in the terminal oligos.

The method for cell-free assembly of nucleic acids comprising assembling DNA fragments to provide a DNA assembly, attaching hairpin oligos to the DNA assembly to form a covalently closed DNA circle, amplifying the DNA circle to form an amplification product, and separating a target product from non-target materials, as discussed above, may provide DNA circles that are resistant to DNA exonucleases. The hairpin oligos may further comprise at least one primer binding site and a restriction enzyme cut site. The method may further comprise digesting the amplification product with a restriction enzyme prior to the separating step to release a plurality of target products. The restriction enzyme may be a DNA base modification-dependent endonuclease.

The present invention may further involve an amplification protocol that seamlessly integrates quality control processes, ensuring the delivery of ready-to-join DNA fragments aligned with a predetermined assembly scheme.

The method for cell-free assembly of nucleic acids comprising assembling DNA fragments to provide a DNA assembly, attaching hairpin oligos to the DNA assembly to form a covalently closed DNA circle, amplifying the DNA circle to form an amplification product, and separating a target product from non-target materials, as discussed above, may further comprise an assembling step comprising assembling a first pool of at least three DNA fragments to form a first assembly product, assembling a second pool of at least three DNA fragments to form a second assembly product, and ligating the first and second assembly product to provide the DNA assembly. The assembling step may comprise introducing a flanking region at the 3′ and 5′end of a DNA fragment (“universal end”). The method may further comprise ligating the target product to another target product.

### BRIEF DESCRIPTION OF THE DRAWINGS

FIG. 1. Shows a diagram for attaching hairpin oligos to a linear nucleic acid molecule to form covalently closed DNA circles.

FIG. 2. diagrams an assembly tree for assembling multiple DNA fragments.

FIG. 3. diagrams an assembly tree for assembling multiple DNA fragments, each DNA fragment comprising a universal end.

FIG. 4. Shows an exemplary negative selection process for removing partially undigested fragments, totally undigested fragments, and the ends of the fragments.

FIG. 5. Shows an exemplary general architecture of universal ends.

FIG. 6. Shows exemplary amplification primers for targeting the universal ends.

FIG. 7. Shows exemplary steps involved in the assembly of molecules.

FIG. 8. Shows another exemplary workflow for the assembly of molecules.

FIG. 9. Shows an exemplary workflow for cell-based clonal selection using dial-out PCR.

### DETAILED DESCRIPTION

The invention provides methods for synthesizing large biopolymers such as genome-scale polynucleotide molecules. In various embodiments, genome-scale polynucleotides are made by attaching together a number of short oligos. The present invention allows for the isolation of sequence-correct DNA fragments from heterogeneous mixtures of fragments through the implementation of several steps for the purification of DNA molecules, the amplification of said DNA molecules, identification of sequence correct DNA fragments (e.g., by Next-generation sequencing (NGS)), and isolation of fragments (e.g., using magnetic beads) as template for PCR-based production of further downstream fragments.

The present invention provides methods for cell-free assembly of nucleic acids. The invention provides certain advantages. DNA assembly products are “cleaned up” by enzymatic steps resulting in an increase in the percentage of sequence correct fragments for “clonal” isolation. Amplification by Rolling Circle Amplification (RCA) produces concatenates simplifying the isolation of sequence correct molecules, as well as hybrid products (e.g., by inclusion of barcode in the hairpin structure). In one embodiment, barcodes are introduced between restriction recognition sites and a downstream cutting position. This would allow for second-strand synthesis in RCA. Present methods allow for the selection of unique priming positions. The introduction of spacers may be used as a barcode as well as a priming site. Obtaining “clonally derived”, sequence verified templates allows for production of high amounts of DNA by high-fidelity PCR amplification. Removal of E. Coli based clonal selection allows for a decrease in the time it takes for the procedure and fully synthetic production of DNA molecules. Cell-free assembly allows for the removal of limitations due to toxic DNA sequences and cloning difficulties. Cell-free assembly allows for the removal of regulatory difficulties and requirements for permits due to the production of gene modified organisms (GMO) in the process of clonal selection.

The present invention provides a method for cell-free assembly of nucleic acids, the method comprising assembling DNA fragments to provide a DNA assembly, attaching hairpin oligos to the DNA assembly to form a covalently closed DNA circle, amplifying the DNA circle to form an amplification product, and separating a target product from non-target materials. The amplifying step may comprise Rolling Circle Amplification (RCA). In a preferred embodiment, hairpins are included in the actual assembly itself, and the last wherein first oligos are self-complementary. The methods may further comprise sequencing the amplification products after the amplifying step. Purification of target products can be achieved enzymatically (Smith and Modrich, 1997) and/or by ligand interactions (Gao and Han, 2001) in which ligands selectively recognize unusual structures of oligo duplexes such as mismatch base pair, bulge, loop, nick, apurinic/apyrimidinic, or deaminated sites. These reagents can be used to assist the removal of impure oligos and impure ligated sequences due to the incorporation of incorrect oligos.

The method may further comprise sequencing of the amplified material to identify molecules that are sequence correct. The inclusion of barcodes in the hairpins allows for ensuring that the Rolling Circle Amplification did not produce hybrid molecules that contain sequences derived from fragments of different origin by cross-hybridization. As noted above, barcodes may be introduced between the modified base of a restriction site and the actual cutting site. This may have particular relevance for second strand synthesis from the RCA forward product.

The current invention also relates to the design of the terminal 5′ and 3′ hairpin oligonucleotides for attaching to the DNA assembly to form a covalently closed DNA circle. For clarification, a preferred closed DNA circle is a continuous self-complementary DNA with no 3′ or 5′ carbon ends (a circular, self-complementary single stranded DNA molecule), as opposed to a double-stranded plasmid.

The terminal 5′ and a terminal 3′ oligonucleotides of a fragment are designed in such a way to have no free 5′ or 3′ ends available. These oligos would enable the production of a covalently closed molecule via ligase enzyme activity. Only the fully closed assembly products would be resistant to DNA exonucleases such as Exonuclease I, Exonuclease III, and Exonuclease V. A cocktail of such enzymes, even in very limited amounts, could enzymatically clean up the sample and deliver a more homogenous solution. This homogenous solution may be directly used as starting material for the amplification reaction resulting in a clean and specific product that does not require further purification.

These hairpin oligonucleotides may further comprise accessory sequences such as a target site for universal PCR primers, attachment moieties such as biotin, or barcoding sequences (e.g., UMI). An attachment moiety may simplify the recovery and downstream handling. The addition of universal primer target sequences and UMI would set up a sequencing-based, cloning-free screening of the produced fragments.

The present invention may further involve an amplification protocol that seamlessly integrates quality control processes, ensuring the delivery of ready-to-join DNA fragments aligned with a predetermined assembly scheme.

Methods for cell-free assembly of nucleic acids comprising assembling DNA fragments to provide a DNA assembly, attaching hairpin oligos to the DNA assembly to form a covalently closed DNA circle, amplifying the DNA circle to form an amplification product, and separating a target product from non-target materials, as discussed above, may further comprise an assembling step comprising assembling a first pool of at least three DNA fragments to form a first assembly product, assembling a second pool of at least a pair of DNA fragments to form a second assembly product, and ligating the first and second assembly product to provide the final DNA assembly. The assembling step may comprise introducing a flanking region at the 3′ and 5′end of a DNA fragment (“universal end”). Such a flanking region can be optimized to prime the amplification of the sequence of interest using a limited set of optimized primers to be selected from a pre-defined and validated library of primers. The flanking region (“universal end”) may be chosen among a library to minimize off-target amplification and enable the enrichment of the desired assembly product. The universal sequences are used for amplification of assembly products for quality control, preparation of fragments for high order assembly, and enrichment of the desired assembly product. The method may further comprise ligating the target product to another target product (repeating the process as many times as desired).

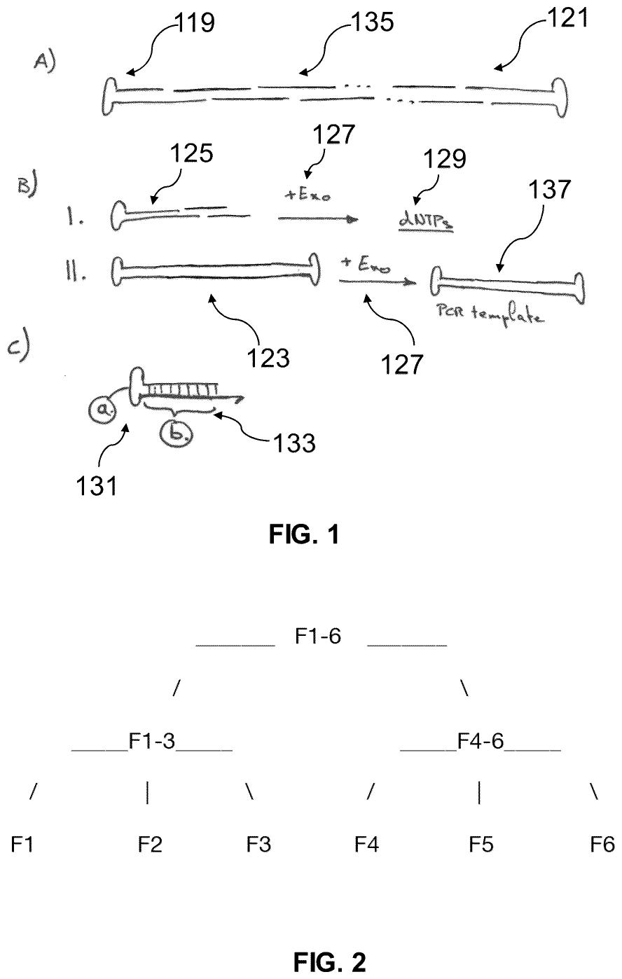

FIG. 1 shows terminal hairpin oligos 119, 121 attaching to a linear nucleic acid molecule 135 via ligase enzyme activity, resulting in a covalently closed DNA circle 123, and incomplete nucleic acid molecules 125. This would confer only to the fully synthetized molecule resistance to DNA exonucleases 127 such as Exonuclease I, Exonuclease III, and Exonuclease V. A cocktail of such enzymes, even in very limited amount could enzymatically clean up the sample and deliver a more homogenous solution to directly use as starting material 137 of the amplification reaction with the aim to obtain a clean and specific product that does not requires further purification. DNA exonucleases would digest incomplete nucleic acid molecules 125 into nucleotides 129. Moreover, these hairpin oligonucleotides could harbor accessory sequences such as a target site for universal PCR (or RCA) primers 131, or barcoding sequences (UMI) 133.

FIG. 2 shows an assembly tree diagramming an assembly process comprising a first pool comprising DNA fragments F1, F2, and F3, and a second pool comprising DNA fragments F4, F5, and F6. The two pools comprising 3 fragments each are assembled, and the resulting products F1-3 and F4-6 are assembled in a new reaction to finish the product resulting in F1-6.

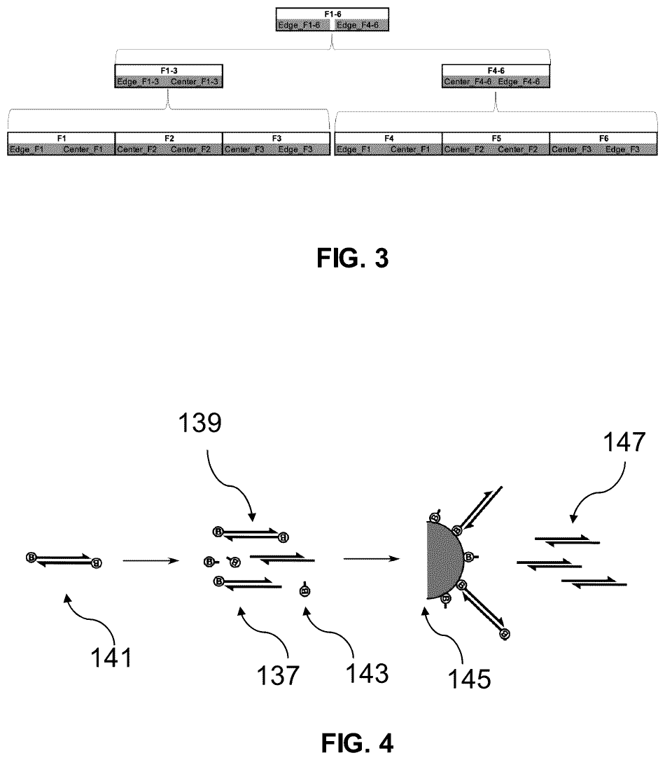

FIG. 3 shows an assembly tree diagramming an assembly process wherein each fragment is assigned two ends (left and right), and each belongs to a specific class of universal ends, depending on its position. (F1 fragment has an Edge-class end on the left side and a Centre-class end on the right side; F2 fragment has two Centre ends and so on and so forth). Edge ends and Centre ends must be different within the pool. As not all assembly processes reach completion, developing a method to easily extract/enrich the desired product is fundamental. This method enables the selective retrieval and enrichment of the target molecule as “F1-3” product can be obtained via PCR of the assembly reaction using the Edge pair of oligos targeting F1 and F3 and no single or intermediate fragments will be amplified. This scheme must propagate along the tree so that assembly product F1-6 shall be retrieved from the assembly using the primer targeting Edge_F1 and Edge_F6.

FIG. 4 an exemplary method of selective enrichment of the desired DNA fragments. Biotinylated DNA fragments 141 go through digestion (e.g. via restriction enzymes such as MspJI, FspEI, LpnPI, McrBC). Partially undigested fragments 137, totally undigested fragments 139, and the ends of the fragments 143 are removed from the reaction using a negative selection procedure (e.g., with streptavidin magnetic beads 145), to only leave the desired DNA fragment 147

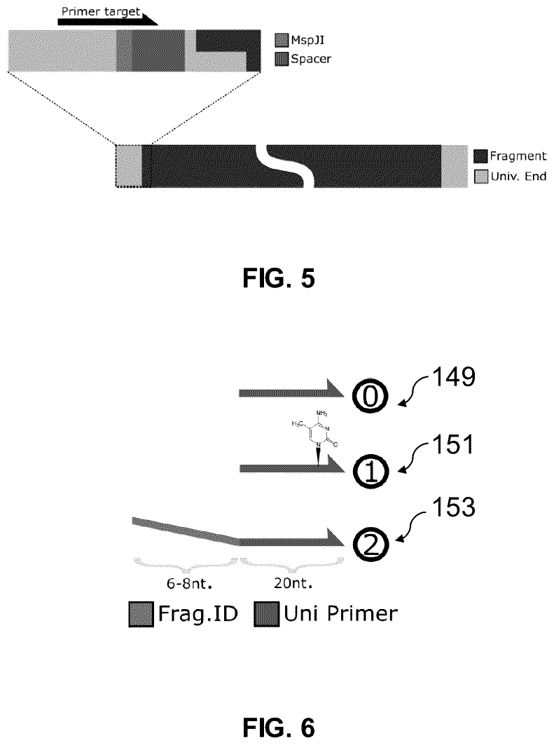

FIG. 5 shows an exemplary general architecture of universal ends comprising and a restriction enzyme site (e.g., for MspJI). The sequence is added on both ends of a fragment and produced during an assembly process. The ends are 20 to 30 nucleotides long comprising 1) 4-8 nucleotides that act as buffer at the very end of the molecule, 2) The primer binding site encompassing the MspJI consensus motif or any other modification-dependent enzyme, and 3) A spacer sequence between the MspJI target and the molecule of interest. Such a spacer can be used to place a secondary restriction site, a fragment identifier or unique molecular identifier (UMI).

FIG. 6 shows exemplary amplification primers that are Unmodified 149, Modified for digestion 151 (e.g., a C-5 methylation (5-mC) or C-5 hydroxymethylation (5-hmC)), and Modified for QC 153.

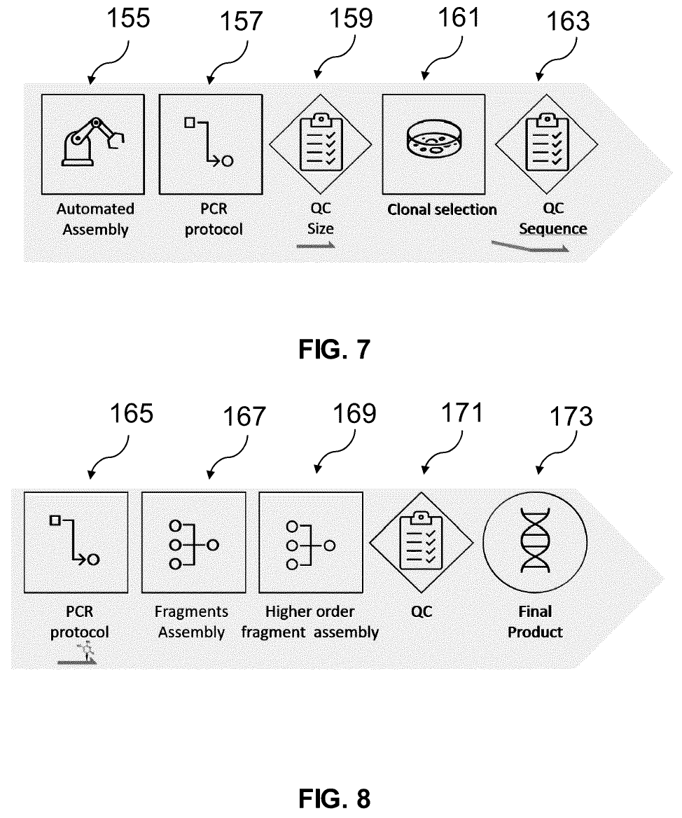

FIG. 7 shows the exemplary steps involved in the assembly of molecules comprising automated assembly 155, PCR protocol 157, QC for size 159, clonal selection 161, then QC for correct sequences 163.

FIG. 8 shows another exemplary workflow comprising the steps of PCR protocol 165, fragment assembly 167, higher order fragment assembly 169, QC 171, then final target product 173. QC 171 verified fragments may be then amplified by PCR using unmodified primers or primers modified for digestion, depending on the ends of the fragment (e.g., Edge, Center, or mixed class). Fragments for the assembly are pooled according to a pre-determined assembly tree. That fragments may then be subject to a protocol combining ends-digestion and ligation of adjacent fragments, lasting about 4 hours with incubations cycling according to the following protocol: 35×(37° C., 2 min+16° C., 5 min)+1×(65° C., 20 min)+1×(4° C., hold).

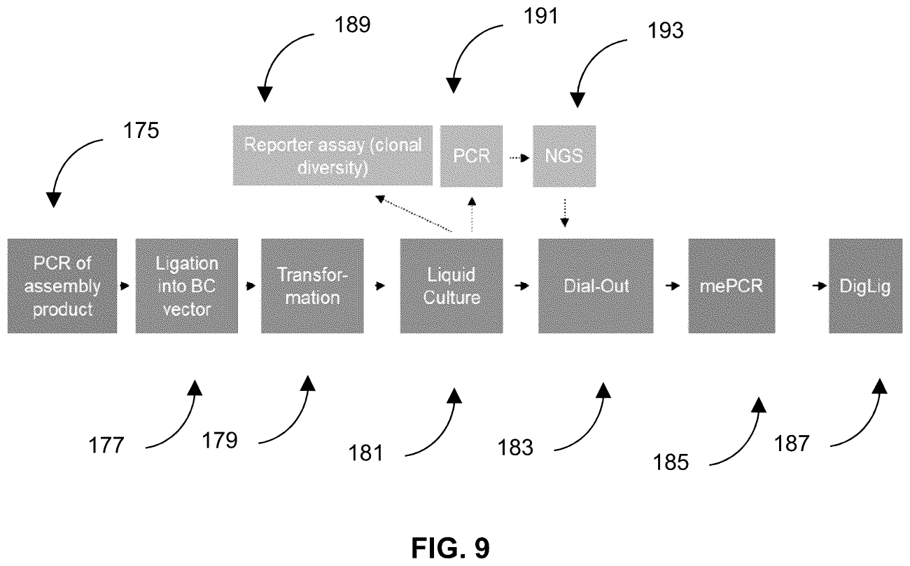

FIG. 9 shows an exemplary workflow for cell-based clonal selection using dial-out PCR, comprising the steps of PCR of assembly product 175, ligation into a vector 177, transformation 179, liquid culture 181, optional reporter assay (i.e., clonal diversity assessment) 189, PCR 191 and NGS 193 for dial-out primer selection, dial-out PCR 183, PCR amplification 185, and digestion and ligation 187. In certain embodiments, the steps are performed in 384 well plates.

In a certain aspect, the assembly of polynucleotides (e.g., DNA) is a hierarchical assembly. Hierarchical assemblies involve attaching different adjacent pairs of DNA fragments from distal locations along the final desired polynucleotide sequence to make an intermediate tier of two-part oligos (e.g., joining 8-mers to form 16-mers), or sub-polynucleotides. Then, the two-part oligos could be joined pairwise (e.g., to form 32-mers). Those joining steps may proceed in a pair-wise fashion to make larger sub-parts of the desired polynucleotide sequence at each tier of assembly. Hierarchical assembly is appealing because it allows many of the steps to be parallelized. To illustrate, when the starting DNA fragments are 8-mers, step 1 produces 16-mers, step 2 produces 32-mers, and step 3 produces 64-mers. The length produced by each step is potentially double the length produced by the prior step and, in theory, the fifteenth step produces a polynucleotide with a length of 131,072 bases. Compared to the 12,500 sequential steps required in the linear approach, a 100,000 base polynucleotide could be assembled with 15 sequential steps by the hierarchical approach. However, successful assembly by the hierarchal approach raises potential issues. For example, if a pair of 8-mer DNA fragments are to be pipetted together from two reaction containers, to correctly form a desired polynucleotide sub-sequence, that pair of oligos must join in the correct order and not to themselves. Thus, the possible combinations of steps available under hierarchical assembly are limited to pairwise combinations in which the free ends of a pair of DNA fragments intended to be joined are biochemically conducive to being joined, and the unintended ends are not biochemically susceptible to being joined.

The polynucleotide molecule may be modified by enzymatic modification, employing any one or more of methyltransferases, kinases, CRISPR/Cas9, multiplex automated genome engineering (MAGE) using λ-red recombination, conjugative assembly genome engineering (CAGE), the Argonaute protein family (Ago) or a derivative thereof, zinc-finger nucleases (ZFNs), transcription activator-like effector nucleases (TALENs), meganucleases, tyrosine/serine site-specific recombinases (Tyr/Ser SSRs), hybridizing molecules, sulfurylases, recombinases, nucleases, DNA polymerases, RNA polymerases or TNases.

DNA assemblies (e.g., polynucleotides) may be purified e.g., by gel electrophoresis, hybrid capture with biotinylated probes, chromatographic, or affinity separation methods. Assembly methods preferably include connecting oligos by a ligation reaction which comprises enzymatic, chemical, or an adaptor ligation. Some embodiments use a ligase, such as any one of a T3, T4 or T7 DNA ligase, or an RNA ligase, a polymerase or ribozymes. Preferably T4 DNA ligase, T7 DNA Ligase, T3 DNA Ligase, Taq DNA Ligase, DNA polymerase, or engineered enzymes are used. Preferably, the following ligation reaction is used: T4 DNA Ligase, at a concentration of 10 cohesive end units per μL supplemented with 1 mM ATP (Sambrook and Russel, 2014, Chapter 1, Protocol 17). Assembly may include hybridizing matching overhangs of a ds DNA fragment or hybridizing a suitable ss oligonucleotide linker. A solid carrier may be used to immobilize one or more of said DNA fragments, the target product (e.g., polynucleotide), or one or more intermediate(s) of assembly. Immobilization may be done with avidin coated beads, by modification of the oligo/poly-nucleotides such as by biotinylation or amino modifications. Modifications may use a surface treated with an amino silane for attachment to 3-aminopropyltrimehtyoxysilane or 3′ glycidoxypropyltrimethoxysilane. Surface chemistries amenable to covalent attachment to nucleic acids include carboxylic acid, an aliphatic amine, aromatic amine, chloromethyl (vinyl benzyl chloride), amide, hydrazide, aldehyde, hydroxyl, thiol, or epoxy, among others. Immobilization/attachment to a solid support may use any of the chemistries described in “Strategies for attaching oligonucleotides to solid supports” by Integrated DNA Technologies, 2014 (22 pages), incorporated by reference.

DNA fragments may be ordered, provided from a library, or produced by a suitable method, such as chemical polynucleotide (or oligonucleotide) synthesis methods, including the H-phosphonate, phosphodiester, phosphotriester or phosphite triester synthesis methods, or any of the massively parallel oligonucleotide synthesis methods e.g., microarray or microfluidics-based oligonucleotide synthesis. The DNA fragments can be produced by any of the enzymatic polynucleotide (or oligonucleotide) synthesis methods e.g., ssDNA synthesis by DNA polymerase terminal transferase, or by reverse transcriptase, which produce hybrid RNA-ssDNA molecules. Specifically, the enzymatic polynucleotide synthesis reaction is performed in vitro. The synthesis reaction may be performed to produce RNA, DNA, xeno nucleic acid (XNA) (which may generally include 1,5-anhydrohexitol nucleic acid (HNA), Cyclohexene nucleic acid (CeNA), Threose nucleic acid (TNA), Glycol nucleic acid (GNA), Locked nucleic acid aka bridged nucleic acid (LNA), Peptide nucleic acid (PNA), Fluoro Arabino nucleic acid (FANA)), or hybrids or any combinations of the foregoing.

DNA fragments may be modified by any one or more of phosphorylation, methylation, biotinylation, or linkage to a fluorophore or quencher. DNA fragments may be capped or blocked to prevent attachment/polymerization to additional nucleotides (e.g., until un-blocked). Suitable blocking or capping chemistries may include those discussed in U.S. Pat. No. 10,041,110; WO 2018/152323; WO 2021/058438; WO 2021/213903; and WO 2021/116270, incorporated by reference. The library described herein may comprise library members which are oligos (e.g., DNA fragments) that can be any or all of the following: unmodified ss; phosphorylated ss; methylated ss; biotinylated ss; phosphorylated, biotinylated and methylated ss; unmodified ds; phosphorylated ds; methylated ds; biotinylated and phosphorylated ds; biotinylated and methylated ds. Preferably, library members comprise a 5′-phosphorylation. Specifically, the library described herein comprises ss oligos comprising fluorophores or quenchers and ds oligos comprising fluorophores or quenchers, locked nucleic acids, and the like.

DNA fragments may be provided in a storage-stable form, preferably a form which is storage-stable for at least 6 months at room-temperature. DNA fragments may be stored in storage containments in a dry state. Dry-state is, for example, achieved by lyophilization, freeze drying, evaporation, crystallization or the like. The enzymes which catalyze the degradation of nucleic acids are typically active at room temperature in a fluid biomolecule preparation. Dry-state storage inhibits such enzymatic activity because such enzymes are generally inactive upon de-hydration and because the degradative chemical reactions which they catalyze typically entail the addition of water (i.e., hydrolysis) of a protein or nucleic acid molecule, thus producing protein or nucleic acid backbone cleavage. In the dry state, there is little or no water (e.g., less than 5%, 4%, 3%, 2% or 1% (w/w) water) as a chemical reactant to support such enzyme catalysis. Additionally, any non-enzymatic hydrolysis of protein or nucleic acid is similarly inhibited, since water is generally unavailable for such reactions.

DNA fragments may include bases such as “A” denoting deoxyadenosine, “T” denoting deoxythymidine, “G” denoting deoxyguanosine, or “C” denoting deoxycytidine, “U” denoting uracil, or other natural nucleosides (e.g. adenosine, thymidine, guanosine, cytidine, uridine), nucleotide-analogs e.g., inosine and 2′-deoxyinosine and their derivatives (e.g. 7′-deaza-2′-deoxyinosine, 2′-deaza-2′-deoxyinosine), azole-(e.g. benzimidazole, indole, 5-fluoroindole) or nitroazole analogues (e.g. 3-nitropyrrol, 5-nitroindol, 5-nitroimidazole, 4-nitropyrazole, 4-nitrobenzimidazole) and their derivatives, acyclic sugar analogues (e.g. those derived from hypoxanthine- or indazole derivatives, 3-nitroimidazole, or imidazole-4,5-dicarboxamide), 5′-triphosphates of universal base analogues (e.g. derived from indole derivatives), isocarbostyril and its derivatives (e.g. methylisocarbostyril, 7-propynylisocarbostyril), hydrogen bonding universal base analogues (e.g. pyrrolopyrimidine), or any of the other chemically modified bases (such as diaminopurine, 5-methylcytosine, isoguanine, 5-methyl-isocytosine, K-2′-deoxyribose, P-2′-deoxyribose). The building blocks are linked by phosphodiester linkage or peptidyl linkages or by phosphorothioate linkages or by any of the other types of nucleotide linkages.

Specifically, the target product (e.g., polynucleotide) has a length of at least 100 base pairs (bps). Specifically, the target polynucleotide has a length of at least 150, 1,000, 10,000 or 100,000 bps or longer. Methods of the invention may include a finalization step e.g., to add one or more nucleotide(s) which correspond to those previously removed from the 3′-end and 5′-end, respectively, to prepare a template of such target ds polynucleotide for the purpose of assembly of the target ds polynucleotide according to a template sequence, such as e.g., to generate blunt ends. Specifically, one or more oligos may be selected for producing blunt ends, which are complementary to any overhang of a prefinal intermediate polynucleotide i.e., complementary to the sticky ends of the polynucleotide. Specifically, respective oligos can be used as primers in a PCR reaction to amplify the final product and to add the remaining oligos to each strand to synthesize the complete target product with blunt ends. The overall goal is to generate blunt end products (scarless—i.e. universal end primer removed). To do so we would digest the final product and blunt using either exonucleases (removing cohesive end) or a polymerase to fill-in.

Specifically, the finalization step comprises a purification step of a target product that has been produced employing standard kits, such as the Monarch PCR & DNA clean up kit from New England Biolabs (product no. T1030), to eliminate remaining DNA fragments, oligos, enzymes, and reagents, thereby obtaining the target product (e.g., ds polynucleotide) as a purified DNA product, ready for further use. Methods may include enriching the target product (e.g., polynucleotide) or one or more intermediates of assembly, by, e.g., polymerase chain reaction (PCR) or rolling circle amplification (RCA). Molecules may be purified by immobilization on a solid phase using a tag, for example a biotin tag, and enrichment using, e.g., PCR amplification. According to a preferred embodiment, two sets of primers are used for target specific enrichment and simultaneous elimination of the tag. Specifically, by using a set of primers specific to the 5′ end of the leading strand and a set of primers specific to the 5′ end of the lagging strand of the polynucleotide that is to be enriched, each comprising a primer that is complementary to at least the overhang and a primer that is complementary to the core sequence of the polynucleotide, the target polynucleotide is amplified without the tag sequence. This has the profound advantage that no additional step is required to remove the tag sequence, e.g., by enzymatic digestion.

In some cases, molecules may be purified by immobilization on a solid phase using a binding pair wherein the probe may comprise a second member of the binding pair. In some cases, probes may be immobilized to a solid surface and the molecules may comprise a first member of a binding pair and the capture probe may comprise a second member of the binding pair. In such cases, binding the first and second members of the binding pair immobilizes the molecule to the solid surface. Examples of binding pair include, but are not limited to biotin-avidin, biotin-streptavidin, biotin-neutravidin, ligand-receptor, hormone-receptor, lectin-glycoprotein, oligonucleotide-complementary oligonucleotide, and antigen-antibody.

Methods may include sequencing the polynucleotide molecule to verify the degree of identity with the desired sequence. Any suitable sequencing method may be used such as pyrosequencing, Illumina sequencing, SOLID sequencing, semiconductor sequencing, DNA nanoball sequencing, Heliscope single molecule sequencing, Single molecule real time (SMRT) sequencing, or Nanopore DNA sequencing. Methods may include restriction or chemical modification e.g., to facilitate cloning the target polynucleotide into a vector or plasmid.

In certain embodiments, the amplifying step includes rolling circle amplification (RCA) or rolling circle transcription (RCT) (see, e.g., Lizardi et al., Nat. Genet. 19:225-232 (1998), which is incorporated herein by reference in its entirety). Several suitable rolling circle amplification methods are known in the art. For example, RCA amplifies a circular polynucleotide (e.g., DNA) by polymerase extension of an amplification primer complementary to a portion of the template polynucleotide. This process generates copies of the circular polynucleotide template such that multiple complements of the template sequence arranged end to end in tandem are generated (i.e., a concatemer) locally preserved at the site of the circle formation. In embodiments, the amplifying occurs at isothermal conditions. In embodiments, the amplifying includes hybridization chain reaction (HCR). HCR uses a pair of complementary, kinetically trapped hairpin oligomers to propagate a chain reaction of hybridization events, as described in Dirks, R. M., & Pierce, N. A. (2004) PNAS USA, 101 (43), 15275-15278, which is incorporated herein by reference for all purposes. In embodiments, the amplifying includes branched rolling circle amplification (BRCA); e.g., as described in Fan T, Mao Y, Sun Q, et al. Cancer Sci. 2018; 109:2897-2906, which is incorporated herein by reference in its entirety. In embodiments, the amplifying includes hyperbranched rolling circle amplification (HRCA). Hyperbranched RCA uses a second primer complementary to the first amplification product. This allows products to be replicated by a strand-displacement mechanism, which yields drastic amplification within an isothermal reaction (Lage et al., Genome Research 13:294-307 (2003), which is incorporated herein by reference in its entirety). In embodiments, amplifying includes polymerase extension of an amplification primer. In embodiments, the polymerase is T4, T7, Sequenase, Taq, Klenow, Tth polymerase, or a Pol I DNA polymerase. SD polymerase, Bst large fragment polymerase, or a phi29 polymerase or mutant thereof. In embodiments, the strand-displacing enzyme is an SD polymerase, Bst large fragment polymerase, or a phi29 polymerase or mutant thereof. In embodiments, the strand-displacing polymerase is phi29 polymerase, phi29 mutant polymerase or a thermostable phi29 mutant polymerase. A “phi polymerase” (or “@29 polymerase”) is a DNA polymerase from the @29 phage or from one of the related phages that, like ϕ29, contain a terminal protein used in the initiation of DNA replication. For example, phi29 polymerases include the B103, GA-1, PZA, ϕ15, BS32, M2Y (also known as M2), Nf, G1, Cp-1, PRD1, PZE, SFS, Cp-5, Cp-7, PR4, PR5, PR722, L17, ϕ21, and AV-1 DNA polymerases, as well as chimeras thereof. A phi29 mutant DNA polymerase includes one or more mutations relative to naturally-occurring wild-type phi29 DNA polymerases, for example, one or more mutations that alter interaction with and/or incorporation of nucleotide analogs, increase stability, increase processivity, enhance accuracy, increase phototolerance, and/or alter another polymerase property, and can include additional alterations or modifications over the wild-type phi29 DNA polymerase, such as one or more deletions, insertions, and/or fusions of additional peptide or protein sequences. Thermostable phi29 mutant polymerases are known in the art, see for example US 2014/0322759, which is incorporated herein by reference for all purposes. For example, a thermostable phi29 mutant polymerase refers to an isolated bacteriophage phi29 DNA polymerase including at least one mutation selected from the group consisting of M8R, V51A, M97T, L123S, G197D, K209E, E221K, E239G, Q497P, K512E, E515A, and F526 (relative to wild type phi29 polymerase). In embodiments, the polymerase is a phage or bacterial RNA polymerases (RNAPs). In embodiments, the polymerase is a T7 RNA polymerase. In embodiments, the polymerase is an RNA polymerase. Useful RNA polymerases include, but are not limited to, viral RNA polymerases such as T7 RNA polymerase, T3 polymerase, SP6 polymerase, and Kll polymerase; Eukaryotic RNA polymerases such as RNA polymerase I, RNA polymerase II, RNA polymerase III, RNA polymerase IV, and RNA polymerase V; and Archaea RNA polymerase.

The amplification step may comprise Rolling Circle Amplification (RCA). DNA-based isothermal techniques include Rolling Circle Amplification (RCA) in which a DNA polymerase extends a primer continuously around a circular DNA template, generating a long DNA product that consists of many repeated copies of the circle. By the end of the reaction, the polymerase generates many thousands of copies of the circular template, with the chain of copies tethered to the original target DNA. This allows for spatial resolution of target and rapid nucleic acid amplification of the signal. Up to 1012 copies of template can be generated in 1 hour. Ramification amplification is a variation of RCA and utilizes a closed circular probe (C-probe) or padlock probe and a DNA polymerase with a high processivity to exponentially amplify the C-probe under isothermal conditions. PCR or rolling circle amplification plus exonuclease digestion of non-circularized material is performed to isolate and amplify the circular targets from the starting nucleic acid pool.

In some embodiments, an endonuclease is or comprises at least one of a restriction endonuclease (i.e., restriction enzyme) that cleaves DNA at or near recognition sites (e.g., EcoRI, BamHI, XbaI, HindIII, AluI, AvaII, BsaJI, BstNI, DsaV, Fnu4HI, HaeIII, MaeIII, NlaIV, NSiI, MspJI, FspEI, NaeI, Bsu36I, NotI, HinF1, Sau3AI, PvuII, SmaI, HgaI, AluI, EcoRV, etc.). Listings of several restriction endonucleases are available both in printed and computer readable forms, and are provided by many commercial suppliers (e.g., New England Biolabs, Ipswich, Mass.). It will be appreciated by one of ordinary skill in the art that any restriction endonuclease may be used in accordance with various embodiments of the present technology. In other embodiments, an endonuclease is or comprises at least one of a ribonucleoprotein complex, such as, for example, a CRISPR-associated (Cas) enzyme/guideRNA complex (e.g., Cas9 or Cpf1) or a Cas9-like enzyme. In other embodiments, an endonuclease is or comprises a homing endonuclease, a zinc-fingered nuclease, a TALEN, and/or a meganuclease (e.g., megaTAL nuclease, etc.), an argonaute nuclease or a combination thereof. In some embodiments, a targeted endonuclease comprises Cas9 or CPF1 or a derivative thereof. In some embodiments, more than one targeted endonuclease may be used (e.g., 2, 3, 4, 5, 6, 7, 8, 9, 10 or more). In some embodiments, an endonuclease may be used to cut at more than one potential target region of a nucleic acid material (e.g., 2, 3, 4, 5, 6, 7, 8, 9, 10 or more). In some embodiments, where there is more than one target region of a nucleic acid material, each target region may be of the same (or substantially the same) length. In some embodiments, where there is more than one target region of a nucleic acid material, at least two of the target regions of known length differ in length (e.g., a first target region with a length of 100 bp and a second target region with a length of 1,000 bp).

According to some embodiments, methods described herein combine cell-based cloning and cell-free dial-out PCR or molecular mining PCR to isolate sequence-correct molecules. This method leverages the advantages of both approaches while mitigating some of their drawbacks. This approach combines bacterial cloning with dial-out PCR to avoid colony picking. Preferred methods involve ligating amplified assembly products into barcoded vectors 177, which may have a unique DNA sequence (e.g., barcode) that can be used to track the original of the DNA molecule and may further include a reporter gene. The ligation products are then transformed 179 into E. coli, introducing the DNA into bacterial cells where it can be replicated, and the transformed cells are grown in liquid culture 181, allowing for amplification of the DNA within the cells. Each cell in the culture represents a clone derived from a single transformation event, and liquid culture is used rather than plating to avoid colony picking. Optionally, a reporter gene can be included in the barcoded vector 189, allowing for the assessment of clonal diversity within the liquid culture by measuring reporter gene expression levels. Then, aliquots of the liquid culture are taken, and the E. coli cells are lysed to release the DNA. PCR amplification 191 is performed directly on the lysates, using primers that target the barcoded vector and, also include dial-out tag sequences, which can be degenerate, partially degenerate, or non-degenerate. The amplified DNA will contain both the assembled sequence and the dial-out tags that will be used for selective retrieval. The PCR products are then subjected to next-generation sequencing (NGS) 193 to identify the sequences and their associated dial-out primer pairs, allowing for selection of error-free molecules. Based on the NGS data, dial-out PCR 183 is performed using primers specific to the dial-out tag sequences flanking error-free molecules. This selectively amplifies the desired, sequence-verified molecules from the mixture generated from the E.coli lysates.

Following dial-out PCR, non-target DNA can be removed through enzymatic digestion if necessary, enhancing the purity of the final product by eliminating unwanted DNA. Dial-out PCR may be performed with end-protected primers to obtain nuclease resistant amplicons. A combination of DpnI, an endonuclease, and an exonuclease cocktail allows for removal of all non-target DNA. At this point mismatch specific endonucleases (e.g. NucS proteins) can be used to further increase the sequence quality (% sequence accurate fragments) of the Dial-Out product. Finally, further PCR amplification 185 and digestion and ligation 187 can be used for assembling larger constructs from the sequence-verified fragments, and according to some embodiments, the amplified and dial-out selected products can be assembled into larger constructs using a variety of assembly methods.

The vectors used in this method may be plasmids, cosmids, or other suitable vectors that can replicate in E. coli. The barcodes may be of any suitable length and sequence, as long as they can be distinguished by NGS. In certain embodiments, the reporters are fluorescent proteins. In other embodiments, the reporters are luminescent proteins. Luminescent proteins are enzymes that convert chemical energy into light and provide high sensitivity. Secreted versions of Luciferases (e.g., Gaussia Luciferase with E. coli secretion tag) allow for measurement from a liquid culture aliquot. The reporter gene used may be luciferase, GFP, β-galactosidase, or any other suitable gene that produces a detectable signal. The PCR primers used for amplification from lysates may be designed to target specific sequences within the vector backbone or the ligated DNA, and the primers for dial-out PCR are designed to target specific dial-out tag sequences. The dial-out tag sequences may be completely degenerate sequences, partially degenerate sequences, or non-degenerate sequences, and the length of the dial-out sequence may vary. The NGS platform used may be Illumina, 454, Torrent, PacBio, or any other suitable platform, and the enzymatic digestion step may use any suitable restriction enzymes or nucleases to remove unwanted DNA.

What is claimed is:

1. A method of cell-free assembly of nucleic acids, the method comprising
ligating amplified nucleic acid assembly products into barcoded nucleic acid vectors;
transforming the amplified nucleic acid into a vector;
culturing the vector in a liquid culture medium;
lysing the vectors to release nucleic acid;
amplifying the nucleic acid with primers that bind to the barcodes and that contain separate tag sequences to produce amplicons;
sequencing the amplicons; and
selectively amplifying the sequenced nucleic acids using primers directed to the separate tag sequences.

2. The method of claim 1, wherein the barcode comprises a reporter.

3. The method of claim 1, wherein the separate tag sequences are dial out tag sequences or molecular mining tag sequences.

4. The method of claim 1, wherein the vector is a bacterial cell or a virus.

5. A method of cell-free assembly of nucleic acids, the method comprising
attaching hairpin oligos to each of the plurality of DNA assemblies to form covalently closed DNA circles including a covalently closed mis-assembled DNA circle;
removing the mis-assembled DNA circle;
amplifying the DNA circles to form amplification products; and
separating target products from non-target materials.

6. The method of claim 5, wherein the separating step comprises (i) capturing, using oligonucleotide probes, the target product onto magnetic beads, or (ii) capturing, using oligonucleotide probes, the target product onto a solid substrate and washing the solid substrate.

7. The method of claim 6, wherein the probes comprise biotin, and the magnetic beads comprise streptavidin.

8. The method of claim 6, wherein the probes comprise glycoproteins, and the magnetic beads comprise lectin.

9. The method of claim 6, wherein the probes comprise a chemical coupling agent that binds to the beads.

10. The method of claim 5, wherein the amplifying step comprises Rolling Circle Amplification (RCA).

11. The method of claim 5, further comprising sequencing the amplification products after the amplifying step.

12. The method of claim 5, wherein each of the hairpin oligos include a barcode.

13. The method of claim 5, wherein the DNA circle is resistant to DNA exonucleases.

14. The method of claim 5, wherein the hairpin oligos further comprise at least one primer binding site and a restriction enzyme cut site.

15. The method of claim 5, further comprising digesting the amplification product with a restriction enzyme prior to the separating step to release a plurality of target products.

16. The method of claim 15, wherein the restriction enzyme is a modification-dependent restriction endonuclease.

17. The method of claim 5, wherein the step of assembling comprises
assembling a first pool of at least three DNA fragments to form a first assembly product;
assembling a second pool of at least three DNA fragments to form a second assembly product; and
ligating the first and second assembly product to provide the DNA assembly.

18. The method of claim 5, further comprising ligating the target product to another target product.

19. A method of cell-free assembly of nucleic acids, the method comprising
assembling DNA fragments to provide a plurality of DNA assemblies including a mis-assembled attaching hairpin oligos to each of the plurality of DNA assemblies to form covalently closed DNA circles including a covalently closed mis-assembled DNA circle;
removing the mis-assembled DNA circle;
amplifying the DNA circles to form amplification products; and
separating target products from non-target materials.

20. The method of claim 19, wherein the mis-assembled DNA assembly comprises a DNA assembly comprising bulges due to mis-ligation.

21. The method of claim 20, further comprising nicking the bulges with an enzyme.

22. The method of claim 21, wherein the enzyme is an endonuclease.

23. The method of claim 22, wherein the endonuclease is mismatch endonuclease I.

24. The method of claim 19, wherein the mis-assembled DNA assembly comprises an incomplete DNA assembly.
